(19) United States
(12) Patent Application Publication
(10) Pub. No.: US 2005/0136016 A1
(43) Pub. Date: Jun. 23, 2005

(54) Hair-relaxing composition comprising at least one base other than hydroxide

(76) Inventors: Gerard Malle, Villiers S/Morin (FR); Damien Lavergne, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/988,562

(22) Filed: Nov. 16, 2004

Related U.S. Application Data
(60) Provisional application No. 60/562,220, filed on Apr. 15, 2004.

(30) Foreign Application Priority Data
Nov. 18, 2003 (FR) 03 50848

(51) Int. Cl. A61K 7/09

(52) U.S. Cl. 424/70.2

(57) ABSTRACT

The disclosure provides ready-to-use cosmetic compositions for relaxing keratin fibers in a cosmetically acceptable medium free of polyhydroxylated alkane, comprising, as a relaxing agent, a base not belonging to the hydroxide family. The disclosure also provides kits comprising compartments to be placed in contact to form the ready-to-use compositions, and processes using these compositions.

This application claims benefit of U.S. Provisional Application No. 60/562,220, filed Apr. 15, 2004, hereby incorporated by reference.

The present disclosure relates to ready-to-use cosmetic compositions for relaxing keratin fibers comprising, as an active relaxing agent, a base not belonging to the hydroxide family. The present disclosure also relates to kits comprising compartments to be placed in contact to form the ready-to-use compositions, and also to processes using the compositions.

The term “keratin fibers,” as used herein, encompasses keratin materials, such as fibers of human or animal origin, such as hair, other body hair, eyelashes, wool, angora, cashmere, fur, and the like. Throughout the specification, reference is made to head hair, although the disclosure is not limited to particular keratin fibers.

The terms “relaxing” and “straightening,” as used herein, include the relaxing, straightening, smoothing-out, and decurling of keratin fibers, for example, Caucasian, Asian, North African, or African hair.

The term “bases not belonging to the hydroxide family,” as used herein, encompasses organic and mineral bases that do not contain hydroxide ions in their chemical formula.

The term “base,” as used herein, means a compound capable of accepting a proton.

The term “polyhydroxylated alkane,” as used herein, means an alkane having from 1 to 20 carbons and from 2 to 15 hydroxyl groups.

The expression “between A and B,” as used herein, means ranging from A to B, the limits A and B being included.

Two techniques may be used to permanently reshape the hair. They are based on breaking the cystine disulfide bonds present in keratin. It will be understood that these techniques can be successfully employed, even if not all of the cystine disulfide bonds are broken.

The first technique comprises opening the disulfide bonds using a composition comprising a reducing agent, optionally rinsing the hair, placing the hair under tension with curlers or the like, or shaping or smoothing by other means, such as smoothing out the hair with a large-toothed comb, with the back of a comb or by hand, and reconstituting the disulfide bonds by applying an oxidizing composition (also known as a fixing composition) to the hair, so as to give the hair the desired shape. This technique makes it possible either to make the hair wavy, or to relax it, straighten it or decurl it.

The second technique comprises performing a lanthionization reaction, using a composition comprising a base belonging to the hydroxide family. This leads to replacement of disulfide bonds (—CH₂—S—S—CH₂—) with lanthionine bonds (—CH₂—S—CH₂—). The lanthionization reaction involves two consecutive chemical reactions:
The first reaction comprises a beta-elimination on the cystine brought about by a hydroxide ion, leading to the breaking of the bond and the formation of dehydroalanine. 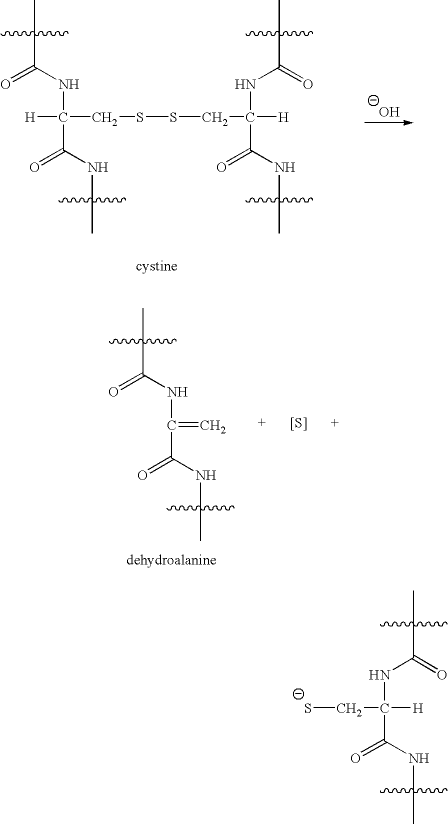
The second reaction comprises a reaction of the dehydroalanine with a thiol group. The double bond of the dehydroalanine formed is a reactive double bond that can react with the thiol group of the cysteine residue that has been released to form a new bond, referred to as a lanthionine bridge or bond or residue.

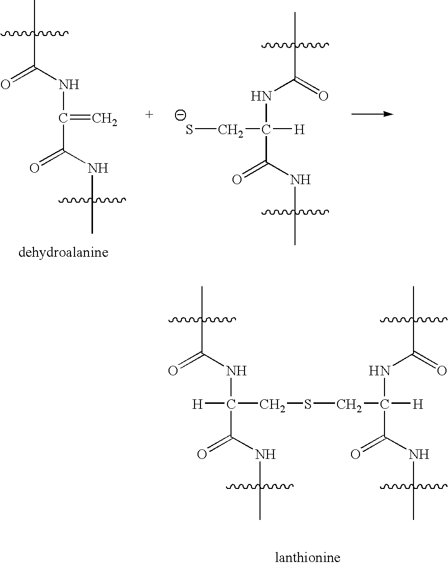

Compared to the first technique, which uses a reducing agent, the lanthionization technique does not require a fixing step, because the formation of the lanthionine bridges is irreversible. It therefore takes place in a single step and makes it possible either to make the hair wavy, relax it, straighten it, or decurl it. However, it is mainly used to relax naturally frizzy or curly hair.

For the first technique, the reducing compositions generally used for the first step of a hair-waving or relaxing operation contain thiols, sulfites, or bisulfites as reducing agents. These agents are generally used in an essentially aqueous medium at concentrations ranging from 0.5M to 1 M in order to obtain good opening of the disulfide bonds. Thiols that may be used include, but are not limited to, thioglycolic acid, cysteamine, glyceryl monothioglycolate, thiolactic acid and cysteine. Thioglycolic acid, for instance, is efficient at reducing the keratin disulfide bonds at alkaline pH, such as in the form of ammonium thioglycolate, and is the product most commonly used in permanent waving (hair waving). However, it has been found that thioglycolic acid must be used in a sufficiently basic medium (in practice, at pH ranging from 8.5 to 9.5) if it is desired to obtain curling or relaxing of sufficient intensity. In addition to the drawback of releasing an unpleasant odor requiring the use of fragrances to mask the odors, the use of a thiol at alkaline pH also may lead to degradation of the fibers and to loss of any artificial coloration.

Sulfites and bisulfites are also used for relaxing. They have drawbacks similar to those of thiols, and may have lower efficacy.

Thiols, sulfites, and bisulfites also may have the drawback of having poor stability in aqueous solution.

In general, the durability of the reshaping effects obtained with thiols and sulfites by reduction of disulfides followed by fixing is considered to be inferior to the effects obtained via the lanthionization technique.

The compositions generally used to perform the lanthionization technique contain, as base, a hydroxide such as sodium hydroxide, guanidinium hydroxide or lithium hydroxide. These lanthionization active agents, which make it possible to open disulfide bonds via a beta-elimination reaction, are generally used in a water-in-oil emulsion at concentrations ranging from 0.4M to 0.6M, by allowing them to act generally for 10 to 15 minutes at room temperature. Sodium hydroxide is the agent most commonly used. Guanidinium hydroxide is also commonly used, and is preferred over sodium hydroxide in many such compositions. Sodium and guanidinium hydroxide are the two most common agents used for relaxing or decurling naturally frizzy or curly hair. They may have several advantages over ammonium thioglycolate and sulfites, including the absence of an unpleasant odor, the fact that only one implementation step is required, resulting in a shorter treatment time, and a much greater durability and efficacy of the reshaping of the hair.

However, these hydroxides have a major drawback of being caustic. This causticity affects the scalp by causing irritation that may be severe. This may be partially overcome by the prior application to the scalp of a greasy protective cream, often referred to as “base” or “base cream”, the word “base” used here not having the meaning of a basic agent in the chemical sense. When the protective cream is combined with the hydroxide in a single composition, this is generally referred to as a “no-base” composition. This “no-base” technology is more convenient.

The causticity of the hydroxides also affects the state of the hair by giving it a coarse feel and making it much more fragile. The fragility may make the hair fray, break, or even dissolve if the treatment is prolonged. In some cases, hydroxides also cause the decoloration of the natural color of the hair.

Formulations containing sodium hydroxide are generally referred to as “lye relaxers” and those not containing it are generally referred to as “no-lye relaxers”.

The “no-lye” relaxing formulations commonly use guanidinium hydroxide. Since guanidinium hydroxide is generally unstable, it is generated extemporaneously by mixing guanidine carbonate and a source of very sparingly soluble hydroxide, such as calcium hydroxide. The reaction between these two compounds results in the formation of guanidinium hydroxide and calcium carbonate, which precipitates in the composition. The presence of this precipitate makes the final rinsing of the hair much more difficult and leaves mineral particles on the hair and scalp that give the hair a coarse feel and an unaesthetic appearance resembling dandruff. The recent preference for guanidinium hydroxide (“no-lye”) over sodium hydroxide (“lye”) relaxing formulations appears to arise from better relaxing efficacy and better skin tolerance. However, these technologies using bases of the hydroxide family are still very aggressive treatments to the hair and scalp and require very strict control of the application time to avoid excessive irritation and impairment of the hair that may go as far as breaking. The aggressiveness arising from the causticity of hydroxides is just reason for these compositions for lanthionization of the hair not to be used for permanent-waving (hair-waving), but instead more often reserved for relaxing (hair-straightening or relaxing).

Furthermore, hydroxides are known to be good agents for hydrolyzing amide functions (see for example, March's Advanced Organic Chemistry, 5th edition, Wiley Interscience, New York, “Hydrolysis of Amides,” pages 474 and seq.), which thus lead to the breaking of peptide bonds by direct nucleophilic attack. The impairments observed in the case of hair and keratin materials in the broad sense may be largely due to partial hydrolysis of the amide bonds of the keratin.

In light of the above problems, there remains a real need for relaxing compositions that are less aggressive to the hair and the skin, including the scalp.

Various studies have been carried out to attempt to simultaneously overcome the drawbacks of reducing agents (used in the first technique) and/or of hydroxides (used in the second technique).

Many reducing agents have been proposed to replace thioglycolic acid, but thioglycolic acid in its ammonium thioglycolate form remains both the reference compound and the compound most widely used in cosmetic formulations, both for shaping and for relaxing and straightening hair.

U.S. Pat. No. 4,530,830 proposes using a composition based on quaternary ammonium hydroxides to replace sodium hydroxide and guanidinium hydroxide and to improve the skin tolerance. However, these compositions are not entirely satisfactory, either in terms of relaxing, or in cosmetic terms.

More generally, many publications describe the combined use of hydroxides, acting as lanthionization active agents, with certain additives generally acting to protect the hair.

Specifically, without using new lanthionization active agents, the improvements proposed relate mainly to the use of additives in order to reduce the damage caused to the hair by hydroxides, for example, in the following applications and patents:
PCT Patent Publication WO 2002/003937, which describes a composition containing C₃-C₅ monosaccharides,
PCT Patent Publication WO 2001/064171, which describes a composition containing complexing agents,
U.S. Pat. No. 5,641,477, which describes a composition containing a hydrogenated starch hydrolysate,
PCT Patent Publication WO 02/085317, which describes a composition containing organic nucleophiles, which react during the second step with the dehydroalanine formed with hydroxides, to give new bridges,
U.S. Pat. No. 5,679,327, which describes a relaxing composition necessarily containing three active constituents for relaxing, namely, an alkali metal hydroxide, an alkaline-earth metal hydroxide and a nitrogenous organic base, each of the constituents being present in the composition in a proportion that would be insufficient to effect relaxing if it was used at this same concentration without the other two active agents. In other words, synergy between three constituents is described, which results in relaxing of the keratin fibers.
   Although all these proposals lead to some improvements, they do not make it possible to sufficiently reduce the damage caused by the actual causticity of the hydroxides.

In relation to the use of hydroxides for relaxing, U.S. Pat. No. 4,524,787 discloses a ready-to-use composition made from a virtually anhydrous “activating” part comprising an organic base in a solvent such as a polyhydroxylated alkane, and an aqueous part having at least 20% of water. However, the latter type of composition is still not satisfactory, either in terms of quality of relaxing, or in terms of mechanical and cosmetic properties of the hair that has undergone this treatment.

As indicated previously, the use of reducing agents leads to mediocre durability for the relaxing or decurling, and the use of hydroxides, because of their causticity, limits their use to the relaxing field.

After extensive studies, it has now been discovered, entirely surprisingly and unexpectedly, that the first step of the lanthionization process can be performed with bases not belonging to the hydroxide family. Excellent results in terms of relaxing, and of cosmetic and mechanical properties of the hair are thus obtained.

Thus, in one aspect, the present disclosure provides cosmetic compositions comprising, in a cosmetically acceptable medium free of polyhydroxylated alkane, at least one base not belonging to the hydroxide family, the cosmetically acceptable medium and the at least one base being chosen such that the base is reactive with the cystines of keratin fibers, via a beta-elimination reaction, to produce dehydroalanine and lead to the formation of lanthionine, to relax the keratin fibers in less than 60 minutes.

The relaxing time may be less than 40 minutes, such as less than 30 minutes.

A beta-elimination active agent resulting in lanthionization that may be used is quinuclidine(1-azabicyclo[2.2.2]octane, Registry Number [100-76-5]).

In the compositions intended for hair relaxing, decurling or straightening processes, the base not belonging to the hydroxide family may be present in a molar concentration ranging from 0.1 M to 4M, which corresponds to concentrations ranging from 1% to 80% by weight, relative to the total weight of the composition, such as a concentration ranging from 0.2M to 4M, which corresponds to concentrations ranging from 2% to 80% by weight, relative to the total weight of the composition.

The pH of the compositions may range from 9.6 to 14, such as from 11 to 13.

In some embodiments, the base not belonging to the hydroxide family is the only relaxing active agent in the composition.

The compositions may also comprise at least one known reducing agent, such as thioglycolic acid or thiolactic acid and ester and amide derivatives thereof, for example glyceryl monothioglycolate, cysteamine and its C₁-C₄ acyl derivatives such as N-acetylcysteamine, N-propionylcysteamine, cysteine, N-acetylcysteine, thiomalic acid, pantethine, 2,3-dimercaptosuccinic acid, sulfites and bisulfites of an alkali metal or alkaline-earth metal, the N-(mercaptoalkyl)-ω-hydroxyalkylamides described in Patent Application EP-A-354,835, the N-mono- or N,N-dialkylmercapto-4-butyramides described in Patent Application EP-A-368,763, the aminomercaptoalkylamides described in Patent Application EP-A-432,000, the N-(mercaptoalkyl)succinamic acid and N-(mercaptoalkyl)succinimide derivatives described in Patent Application EP-A-465,342, the alkylamino mercaptoalkylamides described in Patent Application EP-A-514,282, the azeotropic mixture of 2-hydroxypropyl thioglycolate and of (2-hydroxy-1-methyl)ethyl thioglycolate described in Patent Application FR-A-2,679,448, the mercaptoalkylaminoamides described in Patent Application FR-A-2,692,481, the N-mercaptoalkylalkanediamides described in Patent Application EP-A-653,202, and the formamidine sulfinic acid derivatives described in Patent Publication No. WO 02/39965.

When the compositions comprise at least one reducing agent, the agent is present at a maximum concentration of 20% by weight, for example, from 0.1 to 10% by weight, relative to the total weight of the composition.

The compositions may also comprise at least one known hydroxide, chosen for example from alkali metal, alkaline-earth metal, transition metal, and organic hydroxides, such as sodium hydroxide, potassium hydroxide, lithium hydroxide, rubidium hydroxide, cesium hydroxide, francium hydroxide, beryllium hydroxide, magnesium hydroxide, calcium hydroxide, strontium hydroxide, barium hydroxide, molybdenum hydroxide, manganese hydroxide, zinc hydroxide, cobalt hydroxide, cadmium hydroxide, cerium hydroxide, lanthanum hydroxide, actinium hydroxide, thorium hydroxide, aluminum hydroxide, guanidinium hydroxide and quaternary ammonium hydroxides.

When the compositions comprise at least one hydroxide, the hydroxide may be present in a concentration ranging from 0.01% to 3.5% by weight, such as from 0.05% to 1.5% by weight, relative to the total weight of the composition.

In some embodiments, the compositions contain 0% of base belonging to the hydroxide family, such as alkali metal, alkaline-earth metal, transition metal, or organic hydroxides.

In some embodiments, the compositions comprise from 0 to 50% water, such as from 0 to 30%, or even from 0 to 20% water.

According to certain embodiments, the basic compositions also comprise at least one surfactant chosen from nonionic, anionic, cationic and amphoteric surfactants, such as alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and other hydroxypropyl ether nonionic surfactants.

When the presently disclosed compositions comprise at least one surfactant, the surfactant may be present in a maximum concentration of 30% by weight, such as from 0.5% to 10% by weight, relative to the total weight of the composition.

The presently disclosed compositions may also comprise at least one treating agent of cationic, anionic, nonionic or amphoteric nature to improve the cosmetic properties of the hair, or to attenuate or avoid its degradation.

Suitable treating agents include, but are not limited to, those described in French Patent Nos. 2,598,613 and 2,470,596. It is also possible to use as treating agents linear or cyclic, volatile or non-volatile silicones and mixtures thereof, polydimethylsiloxanes, quaternized polyorganosiloxanes such as those described in French Patent Application No. 2,535,730, polyorganosiloxanes containing aminoalkyl groups modified with alkoxycarbonylalkyl groups, such as those described in U.S. Pat. No. 4,749,732, polyorganosiloxanes such as the polyoxyalkyl polydimethylsiloxane copolymer of the dimethicone copolyol type, a polydimethylsiloxane containing stearoxy end groups (stearoxy dimethicone), a dialkylammonium acetate polydimethylsiloxane copolymer or a polydimethylsiloxane polyalkylbetaine copolymer described in British Patent No. 2,197,352, polysiloxanes organomodified with mercapto or mercaptoalkyl groups, such as those described in French Patent No. 1,530,369 and in European Patent Application EP 295,780, and also silanes such as stearoxytrimethylsilane.

The basic compositions may also comprise other treating ingredients, such as cationic polymers, for instance those used in the compositions of French Patent Nos. 79/32078 (2,472,382) and 80/26421 (2,495,931); ionene cationic polymers, such as those used in the compositions of Luxembourgian Patent No. 83,703; basic amino acids, such as lysine or arginine; acidic amino acids, such as glutamic acid or aspartic acid; peptides and derivatives thereof; protein hydrolysates; waxes; swelling agents, penetrating agents, or agents that reinforce the effectiveness of the reducing agent, such as the SiO₂/PDMS (polydimethylsiloxane) mixture, dimethylisosorbitol, urea and its derivatives, pyrrolidone, N-alkylpyrrolidones, thiamorpholinone, alkylene glycol or dialkylene glycol alkyl ethers, for instance propylene glycol monomethyl ether, dipropylene glycol monomethyl ether, ethylene glycol monoethyl ether and diethylene glycol monoethyl ether, and 2-imidazolidinone; other compounds such as fatty alcohols; lanolin derivatives; active ingredients such as pantothenic acid; agents for preventing hair loss; antidandruff agents; thickeners; suspending agents; sequestering or complexing agents; opacifiers; sunscreens; fragrances; and preserving agents.

In some embodiments, the compositions comprise at least one base not belonging to the hydroxide family that relaxes keratin fibers without being placed in contact beforehand with an organic solvent.

The compositions may be in the form of a thickened cream so as to hold the hair as stiff as possible. These creams are made in the form of “heavy” emulsions, for example based on glyceryl stearate, glycol stearate, self-emulsifying waxes, or fatty alcohols.

Liquids or gels containing thickeners, such as carboxyvinyl polymers or copolymers that “stick” the hairs together and hold them in a smooth position during the leave-in time, may also be used.

The compositions may also comprise at least one adjuvant chosen from silicones in soluble, dispersed or microdispersed form; nonionic, anionic, cationic and amphoteric surfactants; ceramides, glycoceramides and pseudoceramides; vitamins and provitamins, including panthenol; plant, animal, mineral and synthetic oils; waxes other than ceramides, glycoceramides and pseudoceramides; water-soluble and liposoluble, silicone-based or nonsilicone-based sunscreens; nacreous agents and opacifiers; sequestering agents; plasticizers; solubilizers such as lower alcohols, such as ethanol, propanol, and isopropanol; acidifying agents; mineral and organic thickeners; antioxidants; hydroxy acids; penetrating agents; fragrances; and preserving agents.

The present disclosure also relates to kits comprising at least two compartments, one of the compartments (i) comprising, in a cosmetically acceptable medium free of polyhydroxylated alkane, at least one base not belonging to the hydroxide family, the cosmetically acceptable medium and the at least one base being chosen such that the base is reactive with the cystines of keratin fibers, via a beta-elimination reaction, to produce dehydroalanine and lead to the formation of lanthionine, to relax the keratin fibers in less than 60 minutes.

The kits also may comprise an additional composition (ii) for caring for, conditioning, making-up, removing makeup from, protecting, cleansing (washing) keratin fibers.

The compositions of the kits are packaged in separate compartments, containers, or devices, optionally accompanied by suitable, identical or different application means, such as fine brushes, coarse brushes, and sponges.

The disclosure also provides processes for relaxing keratin fibers comprising applying to the keratin fibers a cosmetic composition comprising, in a cosmetically acceptable medium free of polyhydroxylated alkane, at least one base not belonging to the hydroxide family, wherein the cosmetically acceptable medium and the at least one base are chosen such that the base is reactive with the cystines of the keratin fibers, via a beta-elimination reaction, to produce dehydroalanine and lead to the formation of lanthionine, to relax the keratin fibers in less than 60 minutes.

The relaxing time may be less than 40 minutes, or even less than 30 minutes.

In the hair relaxing or decurling or straightening processes described herein, the basic composition is applied to the hair, and the hair is then subjected to mechanical reshaping, giving it a new shape, via an operation of smoothing-out the hair with a large-toothed comb, with the back of a comb or by hand. After a leave-in time of from 5 to 60 minutes, for example, from 5 to 40 minutes, the hair is smoothed out again and is then thoroughly rinsed.

After applying the composition described herein, the head of hair may be subjected to a heat treatment by heating to a temperature ranging from 30° C. to 60° C. In practice, this operation may be performed using a hairstyling hood, a hairdryer, an infrared ray dispenser, and other standard heating devices.

It is also possible to use, as means of both heating and smoothing out the hair, a hot iron at a temperature ranging from 60° C. to 220° C., for example from 120° C. to 200° C.

The disclosure also relates to the use of a base not belonging to the hydroxide family as an active agent for relaxing keratin fibers.

The disclosure also relates to an active agent for relaxing keratin fibers, by means of a beta-elimination reaction, producing dehydroalanine and leading to the formation of lanthionine, comprising at least one base not belonging to the hydroxide family.

The disclosure may be understood more clearly with the aid of non-limiting examples that follow. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific example are reported as precisely as possible. Any numerical value, however, inherently contains certain errors necessarily resulting from the standard deviation found in its respective testing measurements.

### EXAMPLE 1

A simplified relaxing composition was prepared, containing, as a relaxing active agent, quinuclidine at a concentration of 1 M in water. The pH of the composition was 12.5. The composition was applied to naturally curly/frizzy North African hair for 30 minutes at a temperature of 50° C. The hair was efficiently relaxed and soft to the touch.

### EXAMPLE 2

A simplified relaxing composition was prepared, containing, as a relaxing active agent, quinuclidine at a concentration of 2M in water. The pH of the composition was 12.9. The composition was applied to naturally curly/frizzy African hair for 40 minutes at a temperature of 50° C. The hair was efficiently relaxed, easy to comb and style, and soft to the touch.

1. A cosmetic composition for relaxing keratin fibers comprising, in a cosmetically acceptable medium free of polyhydroxylated alkane, at least one base not belonging to the hydroxide family, the cosmetically acceptable medium and the at least one base being chosen such that the base is reactive with at least one cystine of the keratin fibers via a beta-elimination reaction, to produce dehydroalanine and lead to formation of lanthionine, to relax the keratin fibers in less than 60 minutes.

2. The composition according to claim 1, wherein the relaxation time is less than 40 minutes.

3. The composition according to claim 2, wherein the relaxation time is less than 30 minutes.

4. The composition according to claim 1, wherein the at least one base is present in a concentration ranging from 0.1 M to 4M.

5. The composition according to claim 4, wherein the at least one base is present in a concentration ranging from 0.2M to 4M.

6. The composition according to claim 1, wherein the pH ranges from 9.6 to 14.

7. The composition according to claim 6, wherein the pH ranges from 11 to 13.

8. The composition according to claim 1, wherein the composition comprises 0% of base belonging to the hydroxide family.

9. The composition according to claim 1, wherein the composition comprises from 0% to 50% water.

10. The composition according to claim 9, wherein the composition comprises from 0% to 30% water.

11. The composition according to claim 10, wherein the composition comprises from 0% to 20% water.

12. The composition according to claim 1, further comprising at least one adjuvant chosen from silicones in soluble, dispersed or microdispersed form; nonionic, anionic, cationic and amphoteric surfactants; ceramides, glycoceramides; pseudoceramides; vitamins and provitamins; plant, animal, mineral and synthetic oils; waxes other than ceramides, glycoceramides and pseudoceramides; water-soluble and liposoluble, silicone or nonsilicone sunscreens; nacreous agents; opacifiers; sequestering agents; plasticizers; solubilizers; acidifying agents; mineral and organic thickeners; antioxidants; hydroxy acids; penetrating agents; fragrances; and preserving agents.

13. The composition according to claim 1, wherein the at least one base not belonging to the hydroxide family is quinuclidine.

14. A kit for relaxing keratin fibers comprising at least two compartments, wherein one of the compartments comprises a first composition comprising, in a cosmetically acceptable medium free of polyhydroxylated alkane, at least one base not belonging to the hydroxide family, the cosmetically acceptable medium and the at least one base being chosen such that the base is reactive with at least one cystine of the keratin fibers, via a beta-elimination reaction, to produce dehydroalanine and lead to formation of lanthionine, to relax the keratin fibers in less than 60 minutes.

15. The kit according to claim 14, further comprising a second composition for caring for, conditioning, making up, removing makeup from, protecting, cleansing, or washing keratin fibers.

16. A process for relaxing keratin fibers comprising applying to the keratin fibers a cosmetic composition comprising, in a cosmetically acceptable medium free of polyhydroxylated alkane, at least one base not belonging to the hydroxide family, wherein the cosmetically acceptable medium and the at least one base are chosen such that the base reacts with at least one cystine of the keratin fibers, via a beta-elimination reaction, producing dehydroalanine and leading to formation of lanthionine, to relax the keratin fibers in less than 60 minutes.

17. The process for relaxing keratin fibers according to claim 16, further comprising subjecting the keratin fibers to a heat treatment by heating the keratin fibers to a temperature ranging from 30° C. to 60° C.

18. The process for relaxing keratin fibers according to claim 16, further comprising smoothing and heating the keratin fibers with a hot iron at a temperature ranging from 60° C. to 220° C.

19. The process for relaxing keratin fibers according to claim 18, wherein the iron temperature ranges from 120° C. to 200° C.

20. The process for relaxing keratin fibers according to claim 16, wherein the keratin fibers are relaxed in less than 40 minutes.

21. The process for relaxing keratin fibers according to claim 20, wherein the keratin fibers are relaxed in less than 30 minutes.
